(19) United States
(12) Patent Application Publication
(10) Pub. No.: US 2014/0273210 A1
(43) Pub. Date: Sep. 18, 2014

(54) HIGH THROUGHPUT MECHANICAL STRAIN GENERATING SYSTEM FOR CELL CULTURES AND APPLICATIONS THEREOF

(71) Applicant: Board Of Regents, The University Of Texas System, Austin, TX (US)

(72) Inventors: Aaron B. Baker, Austin, TX (US); Mitchell Wong, Dallas, TX (US)

(73) Assignee: Board Of Regents, The University Of Texas System, Austin, TX (US)

(21) Appl. No.: 14/206,452

(22) Filed: Mar. 12, 2014

Related U.S. Application Data
(60) Provisional application No. 61/777,958, filed on Mar. 12, 2013.

(51) Int. Cl. C12M 1/42; C12M 3/00; C12M 1/32; C12N 5/071

(52) U.S. Cl. CPC C12M 35/04; C12N 5/0691; C12M 21/08; C12M 23/12
USPC 435/372; 435/305.2; 435/366

(57) ABSTRACT

The present application relates to an adaptable cell culture system that allows the application of mechanical strain to cells in culture through the displacement of a stretchable cell culture substrate. The system can apply dynamically heterogeneous strains to simulate the complex in vivo strain profiles on cultured cells, the strains including simulation of physiologic waveform such as a simulation of normal or diseased physiological biphasic stretch of the cardiac cycle, a simulation of normal or diseased physiological arterial waveform, or a cyclic mechanical strain. The system is modular and compatible with commercially available cell culture plate formats and robotic liquid handling devices. The system has a variety of applications including screening compounds for cardio toxicity or therapeutic activity, and identifying drug target, all using cell culture under the mechanical strain applied by the system.

### CROSS-REFERENCE TO RELATED APPLICATIONS

This application claims the benefit of U.S. Provisional Application No. 61/777,958, filed Mar. 12, 2013, which is hereby incorporated herein by reference in its entirety.

### STATEMENT REGARDING FEDERALLY SPONSORED RESEARCH OR DEVELOPMENT

This invention was made with Government Support under Grant No. OD008716 awarded by the U.S. National Institute of Health. The Government has certain rights in the invention.

### TECHNICAL FIELD

The present application relates to high throughput mechanical strain generating system for cell cultures and its applications in various aspects of drug discovery.

### BACKGROUND

Cells in the body experience a highly variable and dynamic mechanical environment. Many cell culture assays are performed in the absence of these physiological forces and, consequently, may be missing a fundamental aspect of the in-vivo environment. For instance, arterial mechanotransduction has been the subject of intense experimental and theoretical study that has revealed a variety of mechanisms through which mechanical forces can alter cardiovascular biology. Mechanical forces can interact with cellular structures through the transmission of force to other elements and through transduction to turn mechanical force into a chemical event. For example, mechanical regulation of the binding between integrins and extracellular matrix is a key step in the mechanotransduction processes. Under mechanical forces, integrins have an altered conformation and binding of extracellular matrix, leading to increased interactions with the cytoskeleton/focal adhesions. These processes lead to changes in cytoskeletal arrangement, cell alignment and downstream physiological functions. The search for potential molecular mechanotransducers has revealed a variety of complex and fascinating mechanisms through which stretch- and flow-induced forces can alter arterial biology. These identified mechanisms include activation of integrins and focal adhesions, receptor tyrosine kinases, primary cilia, stretch-sensitive ion channels, and cytoskeletal elements. Although these pathways are known to be involved in sensing forces, much remains to be understood as to how these pathways work together to guide the ultimate biological response or how best to intervene to create therapies for disease.

Systems for applying mechanical stretch to cells in culture have been used for many years. Fundamentally, the vast majority of these devices work on the principle of applying mechanical forces to flexible substrates on which cells can be grown. These systems have fallen into four broad categories including those that apply uniaxial stretch through substrate extension, biaxial strain through substrate bending, biaxial strain through out-of-plane circular substrate distention and biaxial strain through in-plane substrate distension. Among these different configurations, in-plane substrate distension is the only one that produces a uniform strain field. This is essential for controlled studies in which well-defined strains are needed to understand the effect of different types of mechanical stress or to recapitulate the physiologic environment accurately. In-plane substrate distension has been induced on cells through forcing a frictionless piston upward through a flexible culture membrane, by applying pneumatic suction around a platen to a similar culture system or by applying biaxial fraction to a sheet of flexible culture membranes. These and similar systems have allowed the identification of mechanotransduction pathways responsive to cell stretch in a variety of cell types.

However, there are several drawbacks inherent to the design of these devices. First, many of these devices have cultured cells within plates that have non-standard dimensions in comparison to commonly used culture plates. This leads to an incompatibility with fluorescent plate readers, robotic pipetting devices and automated microscopy/quantitation systems. Second, the inherent design of a cam or pneumatic system-based motion places a limitation of the generation of complex, temporal stretch profiles that are present in the real vascular system. The waveform of the cam-based system can only be modified by creating a cam with altered geometry. Thus, it would be cumbersome to pursue studies modifying stretch waveform features in a systematic manner. In addition, the throughput or number of samples that can be treated simultaneously has been a limitation in many systems. This property limits the utility of these systems for studies that might screen compound libraries or the system level behavior of cells under mechanical forces.

### SUMMARY

The present application relates to a system for applying mechanical strain to cell cultures in one or more wells of a culture plate. The system comprises (a) a platen comprising a plurality of pistons, each piston being alignable with a well of the culture plate; and (b) a linear motor in operative communication with the platen wherein said motor is operable to move the platen in a predetermined pattern to cause one or more of the pistons to apply a mechanical force to the one or more wells with which the piston is aligned. The predetermined pattern can be used to apply mechanical strain to one or more wells of the plate based on a physiologic waveform. The physiologic waveform can be a physiologic stretch waveform that simulates cardiac stretch during myocardial contraction, arterial stretch waveforms in vascular beds, mechanical stretch on lung cells during breathing, or stretch on cell of the digestive system including the intestinal cells. The predetermined pattern can be used to apply mechanical strain to one or more wells of a plate based on an arbitrary temporal strain profile. The motor of the system can be capable of generating temporal and complex wave forms that can be transmitted to the cell culture through the mobile platen, the complex waveforms comprising a simulation of normal or diseased physiological biphasic stretch of the cardiac cycle, a simulation of normal or diseased physiological arterial waveform, or a cyclic mechanical strain. The bottoms of the wells of the plate comprise a deformable membrane. In preferred embodiments, the format of the pistons coupled to the platen matches the format of the wells of the plate. When the one or more pistons impact one or more wells, they can displace the bottoms of the wells of the plate to generate variable and dynamic mechanical strain to the wells of the impacted plate. In some embodiments, the membrane of the plate is a silicone based stretchable membrane. The heights of the pistons of the platen are tunable. In some embodiments, the pistons of the platen are of varying or uniform height(s) to impose heterogeneous or uniform mechanical strain(s) to the wells of the plate. The plate used in the system can be a standard 6 (2×3) well format, 96 (8×12) well format, 384 (24×16) well format, or a combination format thereof. Moreover, the platen can be coupled to pistons in matching formats, e.g., 6 (2×3) piston format, 96 (8×12) piston format, 384 (24×16) piston format, or a combination format thereof. The system optionally comprises a supporting structure that secures the placement of the platen and the plate to the system. The platen and/or plate of the system can be modular relative to the supporting structure. The plates of the system are preferably compatible with commercially available robotics for processing, liquid handling, screening, and plate reading.

The present application further relates to a method for applying mechanical strain to cell cultures in a plate. The method comprises applying mechanical strain generated from a motor through matching pistons of a mobile platen to the wells of the plate through displacing the flexible bottom of the wells of the plate through the pistons of the platen. The bottoms of the wells can comprise a deformable membrane. Preferably, the format of the pistons of the platen matches the format of the wells of the plate. The mechanical strain generated to the cell culture in each well can be homogeneous. For example, the uniform mechanical strains can be applied to all the wells of the plate simultaneously through pistons of the platen that have uniform height. The mechanical strain generated to the cell culture in each well can be heterogeneous. For example, the mechanical strains can be applied to all the wells of the plate simultaneously through pistons of the platen that have heterogeneous heights. The mechanical strain can be generated by the motor as a temporal and complex wave form that can be transmitted to the cell culture through the platen, the complex waveforms comprising a simulation of normal or diseased physiological biphasic stretch of the cardiac cycle, a simulation of normal or diseased physiological arterial waveform, or a cyclic mechanical strain. The cell culture can comprise, for example, cardiomyocytes, vascular smooth muscle cells, or stem cells.

The present application additionally relates to a method for screening compounds for cardio toxicity or therapeutic activity using cell culture under mechanical strain. The method comprises applying mechanical strain to cell culture during the screening process.

The present application also relates to a method for genetic screening to identify drug target using cell culture under mechanical strain. The method comprises applying mechanical strain to cell culture during genetic screening. For example, the cells are transduced with Lentivirus constructs before or after being transferred to the wells of the plate. The method disclosed herein optionally comprises exposing the cell culture under mechanical strain to additional physiological influence such as fluid flow.

These and other features and advantages of the present invention will become more readily apparent to those skilled in the art upon consideration of the following detailed description and accompanying drawings, which describe both the preferred and alternative embodiments of the present invention.

### BRIEF DESCRIPTION OF THE DRAWINGS

FIG. 1A is a side view of an embodiment of a cell culture system. FIGS. 1B to 1D are perspective views of an embodiment of a cell culture system.

FIG. 2A is an exploded view of an exemplary culture plate assembly. FIGS. 2B and 2C are an exploded view (FIG. 2B) and perspective view (FIG. 2C) of an exemplary platen with pistons in six by 6-well format.

FIG. 3A is a cross-sectional view of two wells within an exemplary cell culture system. FIG. 3B is a top view of an exemplary cell culture system undergoing radial strain.

FIGS. 4A and 4B are perspective views of exemplary platens with six matching 96-well formatted pistons. FIGS. 4C and 4D are perspective views of exemplary pistons.

FIG. 5A is an image illustrating an exemplary system fully loaded with six E-well plates in a culture incubator. FIG. 5B depicts a stencil that can be used for strain calibration. FIG. 5C is a diagram showing the strain on the membrane as a function of vertical motor displacement. Each 100 counts on the motor index are equivalent to 1 mm of displacement. The error bars at each point represent the variability between 6 wells within the plate (SEM). FIG. 5D is a diagram showing the uniformity of the circumferential strain within the well. Plotted is the circumferential strain at three radii from the center of the well. The total well diameter is 35 mm. FIG. 5E is a diagram showing the alterations in applied strain due to membrane relaxation after 96 hours of cyclic mechanical strain (10% strain, 1 Hz loading and ambient temperature of 37° C.).

FIG. 6A is an illustration of the qualitative nature of pressure waveforms in the arterial system versus those applied by devices with a sinusoidal displacement. Waveforms were taken from human studies of arterial distention. FIG. 6B shows the input position command to linear motor for a brachial-type stretch waveform. FIG. 6C shows the output motor position from motor position sensor. FIG. 6D shows the membrane strain as measured by the displacement of markers on the membrane surface.

FIG. 7 shows a western blot for Sdc-1 in Sdc-1 knockout (KO) cells transfected with lentiviral vectors for wild type (WT) Sdc-1 and mutants.

FIG. 8 is a vascular smooth muscle cells with Sdc-1 knockout and mutated Sdc-1 have increase formation of focal adhesions, actin stress fibers and activation of ERK.

FIG. 9A illustrates a FRET based RhoA sensor. FIG. 9B is an image showing active RhoA in WT and S1KO cells transduced with the fret based RhoA sensor. FIG. 9C is a graph showing FRET signal in the WT and S1KO cells grown to confluence and under mechanical forces.

FIG. 10 shows a Western blot of vSMCs for HPA after transduction with shRNA expressing vectors targeting heparanase or a control scrambled sequence.

FIG. 11 is a graph showing release of lactate dehydrogenase (LDH) by vascular smooth muscle cells in the presence or absence of 10 nM mithramycin with or without mechanical strain.

FIG. 12 illustrates experiments for performing gene screening studies to identify the set of genes involved in the regulation of vSMC phenotype by mechanical force.

FIG. 13 illustrates experiments to examine genes involved in mechanical load medicated regulation of mesenchymal stem cells (MSCs) differentiation.

FIG. 14 illustrates a dual luciferase reporter (Gluc) and constitutive alkaline phosphatase (SEAP) reporter vector for quantifying expression of Nkx2-5 gene transcription in MSCs.

### DETAILED DESCRIPTION

The present invention now will be described more fully hereinafter with reference to specific embodiments of the invention. Indeed, the invention can be embodied in many different forms and should not be construed as limited to the embodiments set forth herein; rather, these embodiments are provided so that this disclosure will satisfy applicable legal requirements.

An adaptable cell culture system is described herein that allows the application of mechanical strain to cells in culture through the displacement of a stretchable cell culture substrate. The system incorporates a high degree of flexibility in the culture format and can apply dynamically heterogeneous strains to simulate the complex in-vivo strain profiles on cultured cells. Experimental analysis of the strains applied to the cell culture substrates of the system described herein demonstrates a high degree of homogeneity in the biaxial strain field in the flexible culture surface. Laser speckle contrast imaging has been used to examine the fluid flow within the culture wells as a result of the applied strain. Cyclic mechanical strain is applied to cultured vascular smooth muscle cells using a sinusoidal waveform typical of previous devices versus arterial distension wave forms found in the aorta, brachial artery and carotid artery of human patients to examine the importance of performing experiments under simulated in-vivo physiological environment.

The system described herein is platform based and consequently is easily adaptable to many standard formats including 6-well format with standard geometry culture plates. The system also incorporates a linear motor as the prime mover and thereby provides a means to apply a variety of arbitrary temporal strain profile for simulating the complexity of the in-vivo mechanical environment and systematically testing strain waveform features. The system can apply uniform strain profiles across the individual wells and addresses the issues of uniformity and repeatability in the multiwell format. The high throughput and temporally tunable system described herein is designed to perform previously difficult studies in a scalable, high throughput manner that can be adapted to studies in vascular biology as well as many fields of mechanical biology in which mechanical stretch plays a role.

The mechanical strain field applied by the systems described herein can have homogeneity such that uniform strain within the majority of the culture well strained area is achieved for examining multiple cells within the strained area and allowing techniques such as western blotting and PCR to be used without consideration for the heterogeneity of the strain field. The mechanical strain field applied by the systems described herein can have selectable strain magnitude and is capable of generating complex strain waveforms in the cell culture. Studies have demonstrated that vascular cells respond to temporal gradients in mechanical forces on strain gradients. The systems described herein therefore are designed to apply a variety of strain profiles within the limits of the acceleration limits of the motor used in the system and the geometry restrictions of the piston. Round shaped pistons are used in the examples disclosed herein to apply uniform biaxial mechanical strain to the membrane of the well bottom. Other shapes such as oval and polygonal can also be adopted for the shape of the pistons. The mechanical strain profiles generated by pistons with these alternative shapes will be based on the aspect ratio of the piston. In general, the cell culture plates of the system described herein are designed to be biocompatible with cell culture. In some embodiments, the cell culture plates of the system described herein are designed to have the same dimension of currently commercially available culture plates such as 6-well plate in 3×2 format, 96-well plate in 12×8 format, or 384-well plate in 24×16 format, which allows the plates from the systems described herein to interface with standard plate reading devices to facilitate the use of many standard and non-standard assays as well as to interface with modern automated cell culture and drug screening instruments. The system can be adapted to other plate format such as 12-well, 24-well, or 48-well formats. Although system described herein is designed to be compatible with commercially available plates and robots, it is understood that the system can be adapted to custom-made plates and robots as well. The system in general is designed to support multiple culture plate formats through modular placement of the plate that can give high throughput and expandability. Although the displacement of the membrane of the system during mechanical strain application creates fluid flow in the wells of the plates, the fluid flow is minimized and/or quantified to calibrate the data of the experiments.

Detailed Designs of the System

In general, the systems described herein comprise three parts: (1) a culture plate with deformable cell culture surface or membrane; (2) a mobile platen comprising low-friction pistons that apply mechanical strain to the cell culture through the membrane; (3) a linear motor operably attached to the platen to provide movement of the platen. The systems in general further comprise a supporting frame that supports the motor, platen and culture plates.

FIGS. 1A to 1D illustrate an embodiment of the disclosed adaptable cell culture system 10. Culture plates 100 can be attached to an immobilized top plate 110 that is connected to a heavy bottom plate 120, for example using hardened support rails 150 and motion rails 160. A platen 130 with pistons 140 can be configured to move vertically along the motion rails 160 on linear bearings. The prime mover in the system can be a hygienically sealed linear motor 200 with position control and fluidic cooling ports. The motor 200 shown in FIGS. 1A and 1B is mounted and stabilized on mounting flanges 190. Springs 180 on the motion rails 160 can provide constant force to reduce load on the motor 200. A perspective view of the culture system 10 is illustrated in FIG. 1B showing six 6-well culture plates 100 with flexible membrane 102 culture surfaces mounted on the top plate 110 of the system. In FIGS. 1A to 1D, the system 10 contains six 6-well culture plates 100 mounted to the top plate 110. This embodiment of the system can use a vertically mounted linear motor 200 to push a platen 130 containing 36 pistons 140 to displace the flexible membrane 102 culture surfaces and create strain on the cells.

Details of the cell culture plate 100 are further illustrated in FIG. 2A. The culture plate 100 can be assembled from a top support plate 103 and bottom support plate 104. The support plates 103, 104 are preferably constructed from a rigid material, such as stainless steel (e.g., 316L) or polycarbonate. The top support plate 103 and bottom support plate 104 each have one or more transverse holes 107 that are aligned when the culture plate 100 is assembled. These holes 107 are sized to allow a size-matched piston 140 to pass through the aligned holes 107 when vertically advanced by the platen 130.

As shown in FIG. 2A, an elastically flexible and deformable membrane 102 (e.g., 0.001 inch thick silicone membrane by Specialty Manufacturing, Saginaw, Mich.) can be sandwiched between the top support plate 103 and bottom support plate 104, such that advancement of the piston 140 stretches the flexible membrane 102 when the piston 140 advances from the hole 107 in the bottom support plate 104 into the hole 107 of the top support plate. Although silicone is used as an example in the plates described herein, other biocompatible stretchable materials known in the art can be used to form the membrane 102. In some embodiments, silicone gaskets 101 can be used to create a seal and prevent leakage. These gaskets 101 also preferably contain holes 107 that align with the holes 107 in the top support plate 103 and bottom support plate 104 when the culture plate 100 is assembled.

A custom-mounting jig can be used to ensure uniform and consistent tension in the membrane when mounted on the plate. The culture plate 100 can then be attached to the fixed top support plate 110 of the system, e.g., using screws. The cell culture plates 100 can be designed to exactly match the dimensions of a standard 6-well, 12-well, 24-well, 49-well, or 96-well plate. This design allows the use of the plates in standard multipurpose plate readers, microscopes and with robotic culture systems. During cell culture experiments, the flexible membrane 102 can be treated with a suitable cell culture coating, such as collagen IV, poly-L-lysine, fibronectin, or combinations thereof. For example, the flexible membrane 102 can be treated with 10 μg/ml type I collagen overnight before cells are seeded into the culture wells. A gas permeable polystyrene lid 105, e.g., from a standard cell culture plate can be used to maintain sterility of the plate. A platen 130 can then be used to support pistons 140 during the motion and displacement of the membrane within the culture plates 100.

As shown in FIGS. 2B and 2C, the pistons 140 can be sandwiched between a platen top plate 131 and a platen bottom plate 132 to form an assembled platen 130. The assembled platen 130 shown in FIG. 2C has 36 individual pistons 140 for use with six 6-well culture plates 100. The platen 130 can be assembled with linear bearings 170 and attached to the system 10 through hardened rods 150 as shown in FIGS. 1A to 1D. These rods 150 can support the vertical motion of the platen 130 and piston 140 and maintain a tight tolerance on the parallel nature of the platen 130 relative to the culture plates 100 and top plate 110 of the cell culture system 10. A central mounting hole 133 in the platen 130 can be included to attach to the linear motor 200 placed underneath the platen 130. The movable platen 130 can then be driven to move up and down vertically. When at rest, the entire platen 130 can be supported by springs 180 attached to the motion rails 160 and held in place with shaft collars. This reduces the static load on the motor 200 and prevents it from dropping when turned off

FIG. 3A illustrates a cross-section of two exemplary wells 108 in a culture plate 100 of the disclosed cell culture system 10 where the culture surface is being deformed by pistons 140 coupled to a platen 130. The side walls of a transverse hole 107 through the top support plate 103 forms the interior walls of the well 108, and the top surface of the flexible membrane 102 forms the bottom culture surface of the well 108. Two pistons 140 are shown coupled to the platen 130 and driven upward through the hole 107 in the bottom support plate 104 into the flexible membrane 102. The flexible membrane 102 is stretched by the piston, thereby increasing the surface area of the bottom surface of the well 108. A standard culture lid 105 (e.g., polystyrene) can be used to maintain sterility in the wells 108. FIG. 3B is a top view of an exemplary 6-well plate, illustrating the direction of stretching in one of the wells 107 of the culture plate 100 after upward advancement of an exemplary piston 140.

FIGS. 4A and 4B are enlarged views of 12×8 arrays of pistons 140 that matches the wells of a 96-well plate. Although the height of the pistons is shown to be uniform in the figures, in some embodiments, a single plate can have pistons 140 of varying heights to apply multiple different strains to wells 108 of a single plate simultaneously. The pistons 140 have a proximal end 146 coupled to the platen 130 and a distal end 145 that contacts the flexible membrane 102 when upwardly advanced. The pistons 140 shown in FIG. 4A have a base 142 and a tip 141 made from different materials. For example, in some embodiments, the piston base 142 is constructed from stainless steel or polycarbonate, while the piston tip 141 is constructed from polytetrafluoroethylene (PTFE). However, as shown in FIG. 4B, the pistons 140 can also be formed from a single material, such as PTFE. As shown in FIGS. 4C and 4D, the piston 140 can have a round cross section (FIG. 4C), a square cross-section (FIG. 4D), or any other suitable shape. However, it is the shape of the distal end 145 surface of the piston 140 determines the direction of mechanical strain on the flexible membrane 102, and therefore on any cells cultured on the flexible membrane 102. For instance, the piston shown in FIG. 4C has a distal end 145 defined by a circular surface, i.e., the cross-section of a hollow cylinder. This circular piston 140 creates radial strain, as show in FIG. 3B. A piston 140 with this shape can have a hollowed portion 144 that extends partially, or completely, from the distal end 145 toward the proximal end 146 of the piston 140. In some embodiments, the piston 140 further includes an orifice 143 along its side that is in fluid communication with the hollowed portion 144. This orifice 143 can vent atmosphere within the hollowed portion 144 to prevent pressure build up within the hollowed portion 144 when the piston 140 is pushing against the flexible membrane 102.

Alternatively, as shown in FIG. 4D, the distal end 146 of the piston can have two linear surfaces that are essentially parallel and configured to contact them flexible membrane 102 on opposite sides of the well 108. This shape can produce strain primarily along a single axis.

The prime mover of the system can be a linear motor 200 such as those produced by Copley Controls (Canton, Mass.). The motor 200 can comprise a central stator that is capable of producing a maximal load of 744 N and continuous load of 215 N. The motor 200 can be hygienically sealed and its feedback position controlled with incremental encoder output and a digital Hall effect sensor. A potential limitation of a linear motor is excess heat produced from the passage of current through the coils, especially if the system is designed to be functional in a standard incubator. The typical culture incubator is designed only to heat from room temperature to a desired temperature (e.g. 37° C.). The linear motor can be encased in a housing that has flow channels within it to allow the circulation of fluid near the coils of the motor. For example, cool water can be cycled through the system using a temperature controlled water bath (VWR). A thermocouple can be used to determine the optimum bath temperature to maintain 37° C. in the well 108. The motor can be mounted to the bottom plate 120 with mounting supports. Support rods can be connected the top plate 110 and bottom plate 120 of the system. Motion rails 160 can also be used to provide support and stability between the top plate 110 and bottom plate 120.

There are several modes for applying strain to silicone membranes for applying loads to cells in culture. These have included in the fluid/pneumatic-based displacement, pin shaped indentation, glass dome indentation. A low-friction based indentation of the membrane can be used in the systems described herein and has been shown to apply nearly homogeneous radial and circumferential strains. In some embodiments a PTFE flange bearing can be used as a piston that created strain on the membrane through upward displacement of the fixed silicone membrane.

The mechanical profiles generated by the system can simulate organ and/or tissue that are stretched during its function or development of normal or pathophysiological processes. In some embodiments, the mechanical strain is a physiologic stretch waveform; an arbitrary temporal strain profile such as a simulation of normal or diseased physiological biphasic stretch of the cardiac cycle, a simulation of normal or diseased physiological arterial waveform, or a cyclic mechanical strain; or mechanical strains that simulate cardiac stretch during myocardial contraction, arterial stretch waveforms in vascular beds, mechanical stretch on lung cells during breathing, stretch on cell of the digestive system including the intestinal cells.

### Application of the System

The dynamic mechanical environment of vascular cells is a powerful regulator of virtually aspect of their behavior. While these effects are widely recognized, the vast majority of studies on vascular cells take place in the absence of the physiological mechanical environment. As a consequence, in vitro studies and assays for drug development lack a critical portion of the in vivo microenvironment and may not realistically correlate with behavior in the body. The system described herein has uniform strain within the piston region, which comprises at least 80% of the total cell culture area in the well. In addition the mechanical displacement of the membrane of the systems described herein creates a more uniform strain outside the piston region (maintaining uniform radial/circumferential strain within 1% to within 3 mm of the well edge.

The piston-based systems described herein also possess better dynamic properties for applying strains of varying frequencies. For the direct mechanical drive of the system described herein, only minimal delay is generated by the force application to the motor by the control system and viscous delays from the membrane material or silicone/PTFE piston interactions. The systems described herein thus can reliably reproduce waveforms with higher frequency content. This is confirmed for sinusoidal frequency testing at 1 Hz for our system in which the actual strains varied less than 1% from the desired strain inputted into the system.

A certain degree of fluid flow in systems that apply loads to flexible culture substrates is unavoidable. In this regard, the systems described herein do not create a significant flow profile over the cells during mechanical loading. With a 5 mm displacement in an 85 mm diameter plate this simple model predicted a peak shear stress of 0.5 dynes/cm² at 1 Hz and 17 dynes/cm² at 10 Hz. Experimental measurement of flow in an embodiment of the systems described herein showed that the fluid flow at 1 Hz of loading frequency and 10% maximal strain was less than 0.3 mL/min suggesting a low magnitude of flow.

In vitro experiment, animal studies and human studies have shown that mechanical stresses are powerful determinants in the regulation of arterial function, myocardial remodeling and the differentiation of progenitor cells in the vascular system. In order to more accurately recreate and parameterize that the complex in vivo environmental it is important to be have precise control over the temporal stretch profiles that are applied to experiments using cultured cells. The system disclosed herein provides a flexible and highly adjustable means to apply stretch to cultured cells by using a linear motor based piston array. The capacity to specify complex strain profiles and alter their character and magnitude independently to other factors enables the systematic characterization of the responses of the cells mechanical environments representing virtually any physiologic or disease state. Thus, the results acquired using the systems described herein is valuable in studying cellular regulation and adaptation to mechanical forces in vivo in many biological systems.

High-Throughput Studies on the Biomechanical Regulation of Cell Phenotype

How mechanical forces regulate the phenotype of cells in the context of cardiovascular disease mechanisms and therapeutic intervention can be investigated using the mechanical strain applied by the systems described herein. Mechanical forces are everywhere in the human experience—from the powerful contractions of heart that drives blood flow to keep people alive to tiny forces and movements that let people sense the lightest touch. At the cellular level, the presence of mechanical forces is also universal and cells are masters of manipulating and sensing miniscule forces to grow, adhere and migrate to carry out their functions. While mechanical forces are necessary and even healthy in many cases, they also play a central role in the diseases that plague society. Numerous studies have highlighted the importance of mechanical forces in cardiovascular disease, cancer, and sensory/neurological disorders. These previous studies not only emphasize a growing body of evidence that mechanical force-mediated control of biology is a ubiquitous phenomenon but also serve to illustrate how little is known about this area in comparison to more established fields. While the tools for advanced genomic analysis have become universal in labs across the country, the field of mechanobiology has languished and has been moved forward only through painstaking traditional studies carried out by specialized laboratories. The primary reason for this slower rate of progress is a lack of practical tools to examine mechanobiology with rigor and efficiency that compares to many modern molecular techniques.

The systems described herein allow, for the first time, large-scale screens of gene function and therapeutic compounds to alter vascular cell phenotype and stem cell differentiation in the presence of highly spatiotemporally controlled mechanical forces. These studies and the technology used can impact a broad spectrum of fields ranging from cardiovascular biology, cancer biology and the study of musculoskeletal disorders. The systems described herein can be used to investigate the development, function, regeneration and reprogramming of cells under physiological and pathological mechanical forces to facilitate a deeper understanding of vascular mechanobiology and provide a library of potential gene targets to help identify therapies for disease. Specifically, the system can be used to investigate the genes controlling the biomechanical regulation of vascular smooth muscle phenotype using a high throughput mechanical loading system and an shRNA gene knockdown library. Additionally, the system can be used to explore and optimize the synergy between mechanical forces and soluble factor/drugs in controlling the differentiation of mesenchymal stem cells into cardiomyocytes.

While mechanical forces are necessary for proper vascular function they are also among the most potent instigators of disease processes. Hypertension, or heightened blood pressure, is the most common clinical diagnosis in the world occurring in over 1 in 4 people worldwide and it is predicted that there will be over 1.5 billion people with hypertension by 2025. Hypertension induces increased forces within the artery during the cardiac cycle and doubles the risk of cardiovascular disease development for each 16% increase in the systolic pressure over the normal range.

Within the artery, vascular smooth muscle cells (vSMCs) compose the bulk of the cellular mass of the vascular wall and are exposed directly to pulsatile variations in pressure leading to cyclic arterial distension and stretch. While vSMC rarely proliferate under normal physiological conditions in adult tissues, they can undergo major phenotypic changes in response to environmental cues such as hypertension, injury and arteriosclerosis. The most well studied shift in phenotype involves vSMCs switching from a contractile phenotype in the healthy artery to a synthetic/proliferative phenotype that drives the formation of disease and arterial fibrosis. Multiple mechanisms act on vSMCs to control phenotype including mechanical forces, angiotensin II, transforming growth factor beta-1 (TGF-β1), reactive oxygen species and many others. The RhoA kinase-dependent activation of serum response factor represents a common pathway for many of the factors regulating vSMC phenotype. Additionally, RhoA has been linked to the mechanical force-mediated regulation of vSMC proliferation through the action of its effectors ROCK and mDia. While it is known that mechanical forces regulate vSMC phenotype, much remains to be understood about the mechanisms, mastor regulatory genes and how multiple pathways may work together. Using the systems described herein, gene screening approach has the potential of identifying new pathways and systematically examining gene function in controlling mechanical force mediated phenotypic regulation of vSMCs. This advance in the field will enable the discovery of new targets for drug discovery and facilitate the study of signaling pathway crosstalk, feedback and system level control mechanisms that are essential to understand how cells interact with their mechanical environment.

Cell therapies using stem cells have emerged as a therapeutic strategy with great potential for treating cardiovascular disease. A major and recurring difficulty in the application of regenerative and tissue engineering strategies in medicine is the control of the differentiation of pluripotent stem cells. Without a deep understanding of the mechanisms of stem cell differentiation, regenerative therapies will remain unsuccessful and will not fulfill their promise in revolutionizing the treatment of disease. The role of mechanical forces in regulating the differentiation of mesenchymal stem cells (MSCs) into cardiomyocytes can be studied using the system described herein. The current known methods for the differentiation of MSCs into functional cardiomyocytes are highly inefficient and are a key limitation in the creation of cell-based based therapies for cardiac repair. The system described herein can be used to perform systematic analysis of the role mechanical force and its interaction with chemical/biological factors in controlling MSC cardiogenic differentiation. The results of this work may increase both basic understanding of MSC biology and provide an optimized set of conditions to enhance cardiogenic differentiation in MSCs.

### EXAMPLES

### Quantification of the Strain on the Culture Membrane

The strain values can be calibrated by measuring changes in radial and circumferential ink marks. Distances between dots along the radial axis were measured to find the radial strain. Circumference changes were measured to find circumferential strain. The dots and circles were marked with an industrial grade permanent pen on a silicone membrane using a customized stencil on paper. The membrane with stencil marks were stretched from base 0 to 500 counts at increments of 100 counts on the machine. At every increment, pictures were taken using Nikon D3100 camera at ‘Micro-Manual’ setting fixed to a stand above the wells. Six pictures were taken per well, resulting in total of 36 pictures. These pictures were converted to TIF file format using Adobe Photoshop. Next, distances between the dots and the circumference on the TIF images were then measured using MetaMorph software by drawing a line between the dots and circles around the circumference marks from the images. The strains were then found by calculating changes in the distance and the circumference relative to the initial 0 count. From this data, count increments were correlated with strain applied by the machine.

Laser Speckle Imaging of Fluid Flow.

Fluid flow in the wells were measured using a laser speckle contrast imaging as described by Argues et al. in Accuracy of the isovolumic relaxation time in the emergency diagnosis of new-onset congestive heart failure with preserved left ventricular systolic function in the setting of b-type natriuretic peptide levels in the mid-range. International journal of cardiology. 2008; 124:400-403. Briefly, a diode laser (785 nm, 50 mW; Thor Labs) was shown upon a well in the mechanical loading device. A Basler 1920×1080 monochrome, CCD with a zoom lens (Zoom7000; Navitar) mounted on microscope boom stand was place vertically over the device and used to record speckle images during mechanical loading. The raw speckle images were converted into speck contrast images using the following relation:

$$K = \frac{{σ}_{S}}{〈 I 〉}$$

where σₛ is the standard deviation and [symbol] I [symbol] is the mean intensity over a 7×7 pixel region of the image. Calibration of flow was performed by imaging flow driven by a syringe pump (Harvard Apparatus) at a known flow rate. Following capture of the raw images the files were processed into laser speckle images using Matlab (MathWorks). The central region of the well was analyzed to avoid shadow artifacts from the laser illumination.

Calibration and Assessment of Strain Uniformity.

An embodiment of the system described herein is shown in FIG. 5A. Both the static and dynamic strains that were produced by the system were assessed using a custom imaging system that consisted of a microscope boom mounted camera. An enlarged view of a custom stamp to create a radial grid pattern in each of the six wells for all six plates attached to the device is show in FIG. 5B. The displacement of the grid elements during the application of load at different piston displacements were tracked and plotted in FIG. 5C and FIG. 5D. Specifically, strain on the membrane as a function of vertical motor displacement is shown in FIG. 5C. Each 100 counts on the motor index are equivalent to 1 mm of displacement. The error bars at each point represent the variability between 6 wells within the plate (SEM). Uniformity of the circumferential strain within the well is shown in FIG. 5D, with the circumferential strain at three radii from the center of the well plotted. The total well diameter is 35 mm. The strain in the wells were also tracked over time during application of 24 hours with a cycle frequency of 1 Hz and a maximal strain of 10% strain. It is found that there was strain relaxation in the system over time that reduced the effective applied strain level in spite of constant maximal displacement. The system can be adjusted to compensate for this change by incrementally increasing the strain over time. This was done by increasing the displacement amplitude over time to maintain constant strain on the material. For a 96-hour experiment the amplitude is increased linearly between that measured at time zero until reaching the appropriate compensated displacement to obtain the same strain at 96 hours. With this compensation, the new strain profile over 96 hours of cyclic loading maintains a constant maximal strain in the loading cycle. FIG. 5E shows alterations in applied strain due to membrane relaxation after 96 hours of cyclic mechanical strain (10% strain, 1 Hz Loading and ambient temperature of 37° C.).

Measurement of Fluid Flow During Strain.

Fluid flow within the wells could be generated due to the motion of the silicone membrane during the mechanical strain application. The shear stress from induced fluid flow could potentially regulate cellular response. Rough order of magnitude estimates have been made to estimate the flow and shear forces resulting from a simplified system in which a membrane in a fluid bath is moved sinusoidally by Schaffer et al. in Device for the application of a dynamic biaxially uniform and isotropic strain to a flexible cell culture membrane. J Orthop Res. 1994; 12:709-719. For a large well of 85 mm in diameter this calculation would put the highest shear stress in the plate to be around 0.5 dynes/cm² located at the edge of the plate for a 1 Hz rate of application with 10% total strain. Higher frequencies of cyclic loading would induce higher shear stresses. An analysis of the fluid flow within the system during application was performed. A laser speckle imaging system was used to characterize the flow on the plate for various strain rates and frequencies of mechanical stretching. Larger fluid volume in the well is shown to correlate with faster flow. Lower frequency applied is shown to correlate with slower fluid flow in the well.

Biological Effects of Stretching Waveform.

Arterial distension waveforms vary throughout the vascular tree and their local effect on vSMC biology in-vivo is unknown. Arterial distension waveforms were measured in human patients for the aorta, brachial and carotid arteries were duplicated as shown in FIG. 6A and scaled to have a maximal strain of 10%. The system could simulate these waveforms by measuring the dynamic strain variation and comparing this to the applied displacement waveform. For example, FIG. 6B shows the input position command of the motor of the system simulates brachial-type stretch waveform. FIG. 6C shows the output motor position from motor position sensor, showing a waveform very similar to the simulated brachial-type stretch waveform from the input command. FIG. 6D shows the membrane strain as measured by the displacement of markers on the membrane surface simulates closely with the input and output brachial-type stretch waveforms shown in FIG. 6B and FIG. 6C, respectively, indicating the system is capable of transmitting complex waveforms to cells in the culture.

PCR Analysis.

Messenger RNA was harvested from the cells following loading using methods described previously and relative mRNA copy number quantified using real time PCT. Real time PCR was used to measure mRNA expression.

Cytotoxicity Assay.

A colorimetric cytotoxicity assay was used assess the presence of cell death during mechanical loading/drug treatment (Promega). This assay measures release of lactate dehydrogenase (LDH) activity and was used according to the manufactures directions. Mechanical stretch to cells in culture using the various waveforms were applied to cell culture. To confirm the biocompatibility of the cells in the system, cell death was measured through an LDH release assay. The maximal strain of 5% was set to be identical between the groups. After 4 hours of mechanical loading the cytotoxicity of the mechanical load was used using a LDH release assay. This analysis demonstrated that there was no significant cell death in the system during mechanical loading.

Quantification of Morphology and Fluorescence Staining.

Following the application of mechanical load, the morphological changes were quantified for changes in cell size, orientation, elongation and fluorescence intensity in the various color channels. Ten images were taken from each well for each experimental group. A minimum of 50 cells was analyzed for each well by outlining the cells using Metamorph image processing software (Molecular Devices, Sunnyvale, Calif.). For the color intensity a background image was taken and subtracted from the fluorescence intensity levels.

Statistics. All results are shown as mean±SEM. A two-tailed student t-test was used to compare two groups in the experiments. An ANOVA with Tukey's post hoc test was used to compare multiple groups of continuous variables. P<0.05 was defined as being statistically significant.

### Example 1

Human vascular smooth muscle cells (Lonza) were grown in DMEM supplemented with 10% fetal bovine serum and antibiotics. The cells were maintained in culture at 37° C. under an atmosphere of 5% CO₂. The cell culture plates with silicone culture surfaces were assembled and sterilized using ethylene oxide treatment. Under sterile conditions, the plates were treated with a solution of 10 μg/ml of type-I collagen (Becton Dickenson, Franklin Lakes, N.J.) for 24 hours. Following collagen coating the plates were washed three times with PBS and the cells passaged onto the plates.

Following application of mechanical strain, the cells were washed twice with PBS warmed to 37° C. The cells were fixed for 10 min in 4% paraformaldehyde. The cells were permeabilized/quenched for 5 min using PBS containing 0.5% Triton X-100 and 20 mM glycine. The cells were then blocked for 40 min with 10% normal goat serum. Primary antibodies to $ were applied overnight at 4° C. in a humidified chamber. The cells were then washed three times with PBS and stained with fluorescently labeled secondary antibody for 90 min at room temperature. The samples were then washed extensively with PBS and coverslipped with anti-fade mounting media containing DAPI (Vector Laboratories, Burlingame, Calif.). The samples were imaged using an inverted fluorescence microscope (Carl Zeiss, Oberkochen, Germany).

### Example 2

Demonstration of cell viability on mechanical loading plates and robust overexpression of genes using lentiviral vectors. As a preliminary experiment mechanical stretch was applied to vascular cells in culture after exposure to a lentiviral delivery system identical to the one that will be used to express the Nkx2.5 promoter reporter gene. In this experiment, cells that were isolated from syndecan-1 knockout mice were treated with lentiviral vectors expressing syndecan-1 (Sdc-1) and mutants of syndecan-1 as shown in FIG. 7. Sdc-1 knockout cells were transformed with lentiviruses to overexpress human Sdc-1 (pSyn1) and Sdc-1 with a deleted cytoplasmic region (pSyn-1-DelC). The Sdc-1 gene was previously cloned into a custom lentiviral system. Mechanical stretch was applied to vascular smooth muscle cells isolated from wild type and Sdc-1 knockout mice for 4 hours of 10% cyclic strain at 1 Hz using the 36-well system described above. These studies demonstrated increased formation of actin stress fibers and focal adhesions in response to mechanical load in the cells without Sdc-1 as shown in FIG. 8. Sdc-1 knockout vSMCs were transformed with lentiviruses to overexpress Sdc-1 (pSyn-1). Mechanical stretching of these cells showed that the focal adhesions did not occur in these cells. These results demonstrate the feasibility of applying stretch to cells on silastic membranes with robust expression of genes through lentiviral delivery.

### Example 3

Demonstration of RhoA Fret biosensor in vascular cells. WT and Sdc-1 KO cells were transduced with a retrovirus expressing a FRET-based RhoA Biosensor. This construct consists of a Rho-binding domain of the rhotekin, which specifically binds to GTP-RhoA, linked to a cyan fluorescent protein (CFP), an unstructured linker, yellow fluorescent protein (YFP), and finally a full-length RhoA. Upon activation, the Rho-binding domain binds RhoA, modifying the relative orientation of the two fluorophores and increasing FRET as shown in FIG. 9A. Using this construct, it was found that Sdc-1 KO cells had a reduction in active RhoA at baseline as shown in FIG. 9B particularly at the ruffle edges of the cells. The cells were grown to confluence and mechanical forces were then applied to the cells. At various time points after initiation of mechanical loading the FRET signal was read in a plate reader and RhoA activity was measured over time. This analysis demonstrated an initial drop in RhoA activity with initiation of mechanical loading followed by increased RhoA activity. In Sdc-1 KO cells the RhoA activation was reduced markedly at longer times as shown in FIG. 9C.

### Example 4

Knockdown of genes using shRNA expressing lentiviral vectors. Library of shRNA expressing lentiviral vectors can be used with the systems described herein to perform gene-screening experiments on vascular cells. These vectors are part of a commercially available library that contains 250,000 shRNA constructs targeting nearly all of the known human genome. As an initial measure of potency of these constructs, lentiviral vectors were made from three constructs that targeted the enzyme heparanase (HPA) and a vector containing a scrambled sequence control. vSMCs were then transduced with these vectors and then the knockdown was assessed using western blotting. The expression of HPA in the cells was significantly reduced compared to the scrambled sequence, confirming the potency and feasibility of this method as shown in FIG. 10.

### Example 5

Toxicity testing in the presence of mechanical load. Toxicity of chemotherapeutic drug mithramycin is tested in the presence or absence of mechanical strain. Specifically, cyclic mechanical load of 5% maximal strain was applied to vascular smooth muscle cells in the presence or absence of 10 nM mithramycin for 24 hours. Experiments of vascular smooth muscle cells in the presence or absence of 10 nM mithramycin without mechanical strains were also performed for comparison during the same time period. Release of lactate dehydrogenase (LDH) was measured as indication of cell death and the results were plotted in FIG. 11. As shown in FIG. 11, in the absence of mechanical strain, the amount of LDH released by control cells and mithramycin are about the same. In contrast, mechanical strain increased the toxicity of the drug leading to a four folds increase in LDH release from the cells compared to the control.

### Example 6

Investigate the genes controlling the biomechanical regulation of vascular smooth muscle phenotype using a high throughput mechanical loading system and an shRNA gene knockdown library. The mechanical forces induced by hypertension and other cardiovascular disorders are important regulators of vSMC biology and phenotype. Using the system described herein a preliminary set of genes responsible for the phenotypic regulation of vSMCs by mechanical load can be identified, which is believed to be a key aspect of arterial biology and a potent determinate of the development and progression of vascular disease.

Referring to FIG. 12, an overview of a method to examine the genes involved in mechanical load-mediated phenotypic regulation of vSMCs is shown. A set of experiments that combine gene knockdown with the high throughput loading system described herein are illustrated. The cells are first passaged onto silastic bottom 96-well plates. The following day they are exposed to lentiviral vectors expressing shRNA targeting a different gene in each well of the plate. These vectors are part of the genome-wide shRNA library developed by the Broad Institute at MIT/Harvard University and now expanded to include over 250,000 shRNA clones covering the entire known human genome (available through Sigma Aldrich Co., St. Louis, Mo.). In each well, a different shRNA clone are transduced into the vSMCs to knockdown gene expression. There are multiple clones for each gene to control for off-target effects, for example four clones per gene can be used.

A set of 143 genes chosen to include 48 genes that are surface receptors, 48 genes related to the cytoskeleton and 47 genes from signaling pathways are examined. Each gene has four shRNA sequences to control for off-target effects. The genes were chosen to include four different scrambled sequences that are used to control for the effect of the virus/vector system. The 143 genes plus scrambled controls give a total of 576 wells, corresponding to the number of samples the system can handle in a single run.

Two loading conditions are applied: one simulates the normal physiological stretch waveform of an artery and a second simulating a hypertensive artery (maximum stretch of 20% strain). In addition, a static culture of the cells without load is kept in the same incubator for comparison. Following mechanical load three outputs are examined, including: (1) RhoA activity, (2) proliferation and (3) gene expression for phenotypic modulation of vSMCs. Experimental details of each of these methods are given below. These outputs are key signaling/phenotypic alterations that are present in hypertension and modified by mechanical forces in the vascular system.

RhoA Activity:

vSMCs previously transduced to express a RhoA activity biosensor as described above are used. This sensor is based on the expression of a synthetic genetic construct that has altered fluorescence resonance energy transfer (FRET) on RhoA activation as shown in FIG. 9. RhoA activity at time zero and at various time points over 24 hours of mechanical loading using a FRET capable plate reader such as Varioskan Flash (ThermoScientific, Waltham, Mass.) are measured, an example of the measurement is shown in FIG. 9C.

Proliferation:

For measuring proliferation mechanical loading are applied to cells for 24 hours and then measure cell proliferation with a high throughput fluorescence-based assay such as Click-iT EdU Cell Proliferation Assay (Invitrogen, Carlsbad, Calif.). After 24 hours of load, the cells are treated with a labeling reagent. Mechanical loading continues another 6 hours and the cells are fixed and permeabilized. The cells are then treated with the detection reagents and fluorescence measured using a plate reader.

Phenotypic Modulation: To measure vSMC differentiation the cells are loaded for 24 hours and then lysed with a lysis buffer containing 1% triton and protease inhibitors. Using automated robotic pipetting ELISA assays are performed for smoothelin and smooth muscle myosin heavy chain (SM-MHC/MHC-11). These assays are kits available through American Research Products, Inc. and LOXO GmbH. Alternatively, “in cell western blot” assays (LI-COR, Inc., Lincoln, Nebr.) can be used to further streamline these measurements.

### Example 7

Explore and optimize the synergy between mechanical forces and soluble factor/drugs in controlling the differentiation of mesenchymal stem cells into cardiomyocytes. Cardiac tissues are normally unable to regenerate after damage from heart attack or myocardial disease due to the inability of cardiomyocytes to proliferate in the adult heart. Bone marrow derived mesenchymal stem cells (MSCs) represent an appealing and promising candidate for establishing effective therapies for cardiac diseases. These cells could be harvested from a patient, differentiated into cardiomyocyte-like cells and reinjected into the damaged heart to provide increase regenerative capacity. Role of mechanical forces in regulating cardiogenic differentiation of human mesenchymal stem cells are defined using the system described herein. These studies can provide both unique insight into the fundamental biology of MSCs and to provide protocol for controlling the cardiogenic differentiation of MSCs for therapeutic application.

Referring to FIG. 13, an overview a method to examine the genes involved in mechanical load mediated regulation of MSC differentiation is illustrated. High throughput assays can be developed and performed to examine the differentiation of MSC to the cardiac cell lines under the influence of both chemical/biological factors and mechanical load. The cardiac transcription factor Nkx2-5 is one of a set of master transcriptional regulators whose expression controls cardiogenesis. A GFP reporter construct containing the promoter region of Nkx2-5 from clone RP11-88L12 from the BACPAC Resource Center at the Children's Hospital Oakland Research Institute was obtained. The construct can be cloned into lentiviral vector expression system demonstrated in FIG. 7. In addition, a luciferase-based reporter lentiviral vector for the Nkx2-5 promoter can be used to transduce validated human mesenchymal stems (Millipore, Billerica, Mass.) with these constructs and select them with puromycin to create a stable line of MSC expressing the Nkx2-5 reporter constructs as shown in FIG. 14.

Using the high throughput mechanical loading system described herein, cyclic mechanical load can be applied to the cells with graded amounts of maximal stretch. This is achieved by having a gradation in the height of pistons that displace the membranes (i.e. taller pistons give higher strain for the same displacement in the forcing platen). Gradation in the height of pistons allows application of multiple levels of strain simultaneously. In this case, all of the cells grown in the top row of the 96-well plate shown receive a maximal stretch of 20% strain each row below receive a lower amount of strain and in the bottom row there is no column and 0% strain is applied as shown in FIG. 13.

The interaction/synergy of mechanical loading with chemical and biological factors that induce cardiogenic differentiation in MSCs are examined. As an initial experiment 5azacytidine (5-aza-C), a molecule well-known to induce murine and human bone marrow cells toward cardiomyocytes lineage is examined. Several other factors that are known to differentiate stem cells in the absence of mechanical load including trans-retinoic acid, dexamethasone, phorbol myristate acetate (PMA), and transforming growth factor beta-1 (TGF-β1) are also examined. A typical experiment involves applying a gradient in mechanical strain (rows on 96-well plate) and a chemical/biological factor dose response as shown in FIG. 13 to explore a wide set of mechanical loads and chemical dose responses to optimize for maximal differentiation of the MSCs. The reporter assay does not require lysing of the cells and thus facilitates repeated measurements on the cells to track changes in MSC differentiation over time.

As used in the specification, and in the appended claims, the singular forms “a,” “an,” “the,” include plural referents unless the context clearly dictates otherwise.

The term “comprising” and variations thereof as used herein are used synonymously with the term “including” and variations thereof and are open, non-limiting terms.

Many modifications and other embodiments of the invention set forth herein will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A system for applying mechanical strain to cell cultures in one or more wells of a culture plate, the system comprising:
(a) a platen comprising a plurality of pistons, each piston being alignable with a well of the culture plate;
(b) a linear motor in operative communication with the platen wherein said motor is operable to move the platen in a predetermined pattern to cause one or more of the pistons to apply a mechanical force to the one or more wells with which the piston is aligned.

2. The system of claim 1, wherein the predetermined pattern is used to apply mechanical strain to one or more wells of the plate based on a physiologic waveform.

3. The system of claim 2, wherein the physiologic waveform is a physiologic stretch waveform that simulate cardiac stretch during myocardial contraction, arterial stretch waveforms in vascular beds, mechanical stretch on lung cells during breathing, or stretch on cell of the digestive system including the intestinal cells.

4. The system of claim 1, wherein the predetermined pattern is used to apply mechanical strain to one or more wells of a plate based on an arbitrary temporal strain profile.

5. The system of claim 1, wherein the motor is capable of generating temporal and complex wave forms that can be transmitted to the cell culture through the mobile platen, the complex waveforms comprising a simulation of normal or diseased physiological biphasic stretch of the cardiac cycle, a simulation of normal or diseased physiological arterial waveform, or a cyclic mechanical strain.

6. The system of claim 1, wherein the bottoms of the wells of the plate comprises a deformable membrane and the format of the pistons of the platen matches the format of the wells of the plate.

7. The system of claim 1, wherein one or more pistons impact one or more wells and displace the bottoms of the wells of the plate.

8. The system of claim 1, wherein the result of one or more of the impacts is the generation of variable and dynamic mechanical strain to the wells of the impacted plate.

9. The system of claim 1, wherein the membrane is a silicone based stretchable membrane.

10. The system of claim 1, wherein the heights of the pistons are tunable.

11. The system of claim 1, wherein the pistons are of varying or uniform height(s) to impose heterogeneous or uniform mechanical strain(s) to the wells of the plate.

12. The system of claim 1, wherein the pistons comprises Polytetrafluoroethylene (PTFE) tips that are smaller in diameter compared to the diameter of the well of the plate.

13. The system of claim 1, further comprising a supporting structure that secures the placement of the platen and the plate to the system.

14. The system of claim 1, wherein the platen and plate are modular relative to the supporting structure.

15. A method for applying mechanical strain to cell cultures in a plate, the method comprising:
applying mechanical strain generated from a motor through matching pistons of a mobile platen to the wells of the plate through displacing the flexible bottom of the wells of the plate through the pistons of the platen, wherein the bottoms of the wells comprises a deformable membrane and the format of the pistons of the platen matches the format of the wells of the plate.

16. The method of claim 15, wherein uniform mechanical strains are applied to all the wells of the plate simultaneously through pistons of the platen that have uniform height.

17. The method of claim 15, wherein heterogeneous mechanical strains are applied to all the wells of the plate simultaneously through pistons of the platen that have heterogeneous heights.

18. The method of claim 15, wherein the mechanical strain generated by the motor is a temporal and complex wave form that can be transmitted to the cell culture through the platen, the complex waveforms comprising a simulation of normal or diseased physiological biphasic stretch of the cardiac cycle, a simulation of normal or diseased physiological arterial waveform, or a cyclic mechanical strain.

19. The method of claim 15, further comprising exposing the cell culture under mechanical strain to additional physiological influence.

20. The method of claim 19, wherein the additional physiological influence is fluid flow.
